(19) United States
(12) Patent Application Publication
(10) Pub. No.: US 2008/0161748 A1
(43) Pub. Date: Jul. 3, 2008

(54) Apparatus and Methods Using Visible Light for Debilitating and/or Killing Microorganisms Within the Body

(76) Inventors: Marc Joshua Tolkoff, Brookline, MA (US); Robert Arcangeli, Westborough, MA (US); Philip Levin, Storrs, CT (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/837,971

(22) Filed: Aug. 13, 2007

Related U.S. Application Data
(63) Continuation of application No. 10/406,137, filed on Apr. 2, 2003, now abandoned.
(60) Provisional application No. 60/369,643, filed on Apr. 2, 2002.

(51) Int. Cl. A61B 18/18

(52) U.S. Cl. 604/21

(57) ABSTRACT

The present invention relates to devices and methods for killing and/or debilitating pathogenic microorganisms, such as the H. pylori bacteria within a patient's body. A light source is provided that emits electromagnetic radiation having wavelengths within the visible spectrum. The light source can be internal and/or external to the patient's body. For embodiments having a light source external to the body, a light guide is provided for transferring electromagnetic radiation from the light source to a location within the patient's body. The light guide has a proximal end optically coupled to the light source and a distal end dimensioned for insertion into a patient's body. A delivery element is also provided to optically couple electromagnetic radiation from the light to a location with a patient's body.

### CROSS-REFERENCE TO RELATED APPLICATIONS

This application incorporates by reference, and claims priority to and the benefit of U.S. Provisional Patent Application No. 60/369,643, filed on Apr. 2, 2002.

### FIELD OF THE INVENTION

This invention relates to apparatus and methods for debilitating and/or killing microorganisms on or within a patient's body and, more particularly, to apparatus and methods for debilitating and/or killing microorganisms on or within a body cavity of a patient using visible light.

### BACKGROUND OF THE INVENTION

Infections involving the human gastrointestinal tract are extremely common, involving many millions of people on an annual basis. These infections include bacteria, viruses, and fungi, and are responsible for significant illness, morbidity and death.

One of the most common gastrointestinal infections in the world is due to Helicobacter pylori (H. pylori), a bacterial pathogen that infects the stomach and duodenum. In industrialized nations, such as United States, H. pylori may be found in 20% or more of the adult population. It is a chronic gut infection and, once acquired, is notoriously difficult to eradicate. Although most infectious bacteria can be readily destroyed by the human immune system, H. pylori is relatively resistant to a host immune response, even if vigorous. At least one reason for H. pylori 's resistance relates to its residing within the lining of the stomach and on the surfaces of the stomach and duodenal cells.

H. pylori is typically a silent infection in humans, often causing a relatively innocuous gastric inflammation or gastritis. In a significant minority of infected people, however, H. pylori can cause more serious conditions including symptomatic gastritis, gastric ulcer, duodenal ulcer, gastric cancer, and gastric lymphoma. The organism is believed to be responsible for approximately 90% of all reported duodenal ulcers, 50% of gastric ulcers, 85% of gastric cancer, and virtually 100% of gastric lymphoma.

Millions of Americans have symptomatic gastritis or the more serious conditions noted above, which are largely due to H. pylori. H. pylori is responsible for thousands of deaths in the United States due to complicated ulcer disease and cancer, and is considered to be a Class 1 carcinogen by the World Health Organization, in the same class as Benzene and DDT.

The organism is found in all countries of the world, causing the same symptoms, diseases, and even deaths, but it is more prevalent in undeveloped countries, presumably due to poor hygiene, contaminated water supplies, and crowding. In Peru and other South American countries, for example, the prevalence rate of H. pylori infection approaches 90%.

Unfortunately, a vaccine is not yet available for H. pylori and, despite years of intensive effort, none is anticipated in the foreseeable future. Difficulties may be due in part to the ineffectiveness of the host's immune response in eradicating H. pylori in even the best of cases. The most common treatment currently available is prolonged and complicated antibiotic regimens involving three or four expensive drugs given over a two-week period. Even using a vigorous antibiotic regimen, 20% or more of those treated are not cured of their infection.

Further, the antibiotics used are powerful, sometimes not well tolerated, and can cause nausea, an altered taste sensation, and diarrhea. Allergic reactions to the antibiotics are not uncommon. In addition to the problems of efficacy and side effects, antibiotic resistance by this organism is growing rapidly. Up to 50% of the H. pylori isolates are now resistant to one or more of the best antibiotics known to cure the infection. The problem of antibiotic resistance is only expected to grow in the future, leading to worsening disease outcomes and an ever-increasing health expense. Thus, a great need exists for a new, effective, rapid and well-tolerated cure of H. pylori, a luminal infection of the gut. There also exists a need for a well-tolerated and effective treatment to debilitate and/or kill microorganisms with as little negative effect as possible on other parts of the body.

### SUMMARY OF THE INVENTION

The present invention solves the problem of effectively treating H. pylori, by taking advantage of H. pylori 's residing within the lining of the stomach and on the surfaces of the stomach and duodenal cells, by providing a visible light treatment. While the invention has utility in destroying microorganisms in various parts of the body, e.g., the mouth, the stomach, bowel, lungs, peritoneal cavity, urinary tract, nasal cavity, ear canal, etc., it is particularly useful in the treatment of gastrointestinal infections. This invention provides a treatment method and apparatus for debilitating and/or killing H. pylori or other microorganisms within a patient's body and is especially suited for treating stomach or duodenal ulcers. The present therapeutic method involves the use of visible light for eliminating pathogenic microorganisms within or supported upon the lining of a body cavity of a patient, e.g., the stomach.

In one aspect, the invention relates to a device for killing or debilitating pathogenic microorganisms within a patient's body. The device includes a light source external to the body emitting electromagnetic radiation having wavelengths within the visible spectrum. The device further includes a light guide having a proximal end optically coupled to the light source and a distal end dimensioned for insertion into a patient's body. The light guide transfers electromagnetic radiation having wavelengths within the visible spectrum from the light source to a location within the patient's body. The device further includes a delivery element optically coupled to the distal end of the light guide for directing electromagnetic radiation transferred thereby to a location with a patient's body. Generally, the device is adapted for killing and/or debilitating microorganisms, including bacteria, such as H. pylori bacteria.

In one embodiment, the light source emits electromagnetic radiation having wavelengths within both the visible and the ultraviolet spectra. The light source can be selected from the group consisting of a laser, a laser diode, a light emitting diode, a lamp, and combinations thereof. The lamp can be selected from the group consisting of an incandescent lamp, a florescent lamp, an arc lamp, and combinations thereof.

In some embodiments, an adapter optically couples the light from the light source to the proximal end of the light guide. The adapter can be selected from the group consisting of a lens, a prism, a mirror, a fiber optic splice, an N-to-1 optical coupler, a connector, and combinations thereof. The light guide can be selected from the group consisting of single strand fiber optic cable, multi strand fiber optic bundle, a gas-filled channel, a fluid-filled channel, a sequence of reflectors, and combinations thereof. The delivery element can be selected from the group consisting of a lens, a prism, a mirror, a balloon, gas, liquid, fluid sprays, fiber fountains, frustrated total internal reflection pads, adhesive optically transmissive coatings, applied optically active materials and combinations thereof.

In another aspect, the invention relates to a method for killing or debilitating pathogenic microorganisms within a patient's body. The method includes providing a light source external to the body, the light source emitting electromagnetic radiation having wavelengths within the visible spectrum. The method also includes optically coupling the electromagnetic radiation into a light guide and directionally coupling electromagnetic radiation from the light guide to a location with a patient's body. In one embodiment, the method is adapted to kill and/or debilitate H pylori bacteria.

In one embodiment, the method includes providing a light source emitting electromagnetic radiation having wavelengths within the visible and the ultraviolet spectra. In another embodiment, the method further includes enlarging the size of a location within the patient's body. The location within the patient's body can be expanded by inserting an expanding element selected from the group consisting of a gas, a fluid, a mechanical support, a balloon, and combinations thereof.

In another aspect, the invention relates to an apparatus for killing or debilitating pathogenic microorganisms within a patient's body, the apparatus including a light source dimensioned for insertion into a patient's body. The light source emits electromagnetic radiation having wavelengths within the visible spectrum. The device further includes a delivery element optically coupled to the light source, for delivering a portion of the coupled visible light to a location within a patient's body.

The light source can be selected from the group consisting of a laser diode, a light emitting diode, an incandescent lamp, a florescent lamp, an arc lamp, and combinations thereof. In one embodiment, the device further includes an energy source located external to the body, whereby the energy source energizes the light source.

In one embodiment, the device includes a tether coupled between the light source and the energy source for coupling energy therebetween. The energy source can be selected from the group consisting of a battery, a power supply, a capacitive storage circuit, an electrical transformer circuit, electromagnetic radiation, beamed electromagnetic energy, beamed acoustical energy, and combinations thereof. In another embodiment, the delivery element is packaged together with the light source.

In yet another aspect, the invention relates to a method for killing or debilitating pathogenic microorganisms within a patient's body, the method including the steps of providing a light source dimensioned for insertion into patient's body, the light source emitting electromagnetic radiation having wavelengths within the visible spectrum; energizing the light source; and directionally coupling at least a portion of the emitted electromagnetic radiation to a location containing pathogenic microorganisms within a patient's body. In one embodiment, the method is adapted for killing and/or debilitating H. pylori bacteria. Further, the location within the patient's body includes at least a portion of a naturally-occurring body cavity. In one embodiment, the light source emits electromagnetic radiation having wavelengths within the visible and the ultraviolet spectra.

In still another aspect, the invention relates to an apparatus for killing or debilitating pathogenic microorganisms within a patient's body, the apparatus including a light-emitting material emitting electromagnetic radiation having wavelengths within the visible spectrum and means for directing at least a portion of the light-emitting material to a location containing pathogenic microorganisms within a patient's body. The light-emitting material can be selected from the group including phosphorescent liquid, chemiluminescent compounds, sonoluminescent compounds; microwave-activated compounds, fluorescent materials, and combinations thereof.

In another aspect, the invention relates to a method for killing or debilitating pathogenic microorganisms in the treatment of an infectious ailment within a patient's body, the method including providing a light-emitting material emitting electromagnetic radiation having wavelengths within the visible spectrum; and delivering at least a portion of the light emitting material to a target area containing pathogenic microorganisms within a patient's body. The target area can includes at least a portion of a naturally-occurring body cavity.

In still another aspect, the invention relates to a method for killing or debilitating H. pylori within a patient's stomach, the method including providing a light source emitting electromagnetic radiation having wavelengths within the visible spectrum and optically coupling the electromagnetic radiation from the light source to a location within a patient's stomach.

This technique can also be used for debilitating surface microorganisms such as Acnes vulgaris and other microorganisms as will be apparent to those skilled in the art. These and other objects, along with advantages and features of the present invention herein disclosed, will become apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a schematic diagram of an external light source embodiment shown treating the inside of a patient's stomach;

FIG. 2 is a schematic diagram of an alternative embodiment of an external light source having multiple light sources;

FIGS. 3A and 3B are schematic diagrams of alternative embodiments of the delivery element of FIGS. 1 and 2, for use with hydrodynamic light guides;

FIGS. 4A and 4B are schematic cross-sectional diagrams of alternative embodiments of the delivery elements of FIGS. 1 and 2;

FIG. 5 is a schematic cross-sectional view of a light source adapted for insertion within a patient's body;

FIGS. 6A and 6B are schematic diagrams of tethered and untethered, respectively, light sources dimensioned for insertion within a patient's body;

FIGS. 7A and 7B are a schematic diagrams of a linear and helical, respectively, light source arrays dimensioned for insertion within a patient's body;

FIGS. 8A-8C are a schematic diagrams of alternative embodiments of the delivery elements of FIGS. 1 and 2;

FIG. 9 is a schematic diagram of another alternative embodiment of the delivery elements of FIGS. 1 and 2, including an insertable diffusing liquid;

FIG. 10 is a schematic diagram of one embodiment of a fiber optic delivery element used with the inventions of FIGS. 1 and 2;

FIGS. 11A-11C are a schematic diagrams of alternative embodiments of the delivery elements of FIGS. 1 and 2;

FIG. 12 is a graph showing test results measuring the effectiveness of H. pylori treatment versus light intensity;

FIGS. 13A and 13B are schematic cross-sectional views of a delivery element including a balloon positioned within a patient's stomach;

FIG. 14 is a schematic cross-sectional view of an inflation lumen;

FIG. 15 is a schematic cross-sectional view of a tethered light source including a balloon positioning element;

FIG. 16 is a schematic cross-sectional view of an alternative embodiment of the inflation lumen shown in FIG. 14;

FIG. 17 is a schematic cross-sectional view of an embodiment using an endoscope inserted through a patient's esophagus;

FIG. 18 is a schematic cross-sectional view of an alternative application for treating the lower digestive system; and

FIGS. 19A-19C are a schematic end, side, and perspective views, respectively, of on embodiment of the invention inserted within an endoscope.

### DETAILED DESCRIPTION OF THE INVENTION

The therapeutic method in accordance with the present invention is suited for use within a patient's body for killing and/or debilitating pathogenic microorganisms, such as H. pylori bacteria. For example, the present invention can be used within various naturally occurring body cavities including, but not limited to, the stomach, the bowel, the lungs, the peritoneal cavity, the urinary tract, nasal cavities, and ear canals. The present invention can also be used to treat other interior locations within a patient's body, such as those accessed and/or created during a surgical procedure (e.g., a muscle). Various devices, fabrication techniques, arrangements, systems and methods of employment, are adapted to illuminate the walls of various body cavities and/or other interior sites within a patient's body. In particular, the illumination includes electromagnetic radiation having wavelengths in the visible light spectrum (i.e., visible light), principally violet/blue light at a sufficient dosage to debilitate and/or kill microorganisms, such as the H. pylori bacteria.

In one embodiment, a light administering device irradiates bacteria and/or other microorganisms with visible light, thereby producing a desired effect of killing and/or debilitating a substantial percentage of the microorganisms, while leaving other tissue and organisms undisturbed.

In one embodiment of a light administering device, a fiber optic device transmits light from an intense external source to the microorganisms living inside a patient's body. In an illustrative example provided herein, body cavity is the patient's stomach and the H. pylori bacteria resides on and/or within at least a portion of the columnar epithelial lining of the walls of the stomach. In general, the treatment disclosed herein can be applied to microorganisms residing on and/or in the epithelium of any other passage or lumen. During treatment, electromagnetic radiation having wavelengths in the visible spectrum (i.e., visible light) reacts with naturally produced or concentrated “endogenous” chromophore, typically a form of porphyrins in the bacteria. In at least one advantageous effect, the light in combination with the porphyrin produces necrosis or cell death evidenced by the microorganism's inability to divide. This may be due, in part, to the excited porphyrins releasing free radicals including oxygen that damage the bacteria and result in necrosis. An advantage of the invention lies in the fact that few organisms and few human cells are sensitive to visible light, so the microorganism being treated (e.g., H. pylori) can be killed without substantially damaging the surrounding tissue. Accordingly, the bacteria can be killed by visible light mediated necrosis without serious destruction of the host cells.

For H. pylori, the endogenously produced porphyrins have a very strong absorption peak in the 405+/−25 nanometer (nm) range, with smaller peaks at about 505, 550, 570, and 655 nm. Light delivered in these narrow wavelengths or in a broad band including the wavelengths of these absorption peaks, e.g., 400-650 nm, and at a sufficient dosage kills and/or debilitates the bacteria, without added drugs or chemicals. The treatment is most effective along the surface, but can also be effective beneath the surface, generally having decreasing benefit with increasing penetration into the body tissue. The penetration of light into tissue varies with wavelength, with greater penetration occurring at longer wavelengths. For example, light at a wavelength of 400 nm penetrates approximately 1 millimeter (mm) or so, while 650 nm light penetrates approximately 3 mm or more. Thus, the wavelength of light can be selected to optimize a desired depth of penetration. Notwithstanding the depth of penetration, a particularly effective wavelength for killing and/or debilitating H. pylori is approximately 400 nm. Additionally or alternatively, using electromagnetic radiation having multiple wavelengths within the visible spectrum (i.e., multicolored light) can be used to provide both effective and deeper therapeutic effect. Total eradication of the microorganism can be claimed with a 2-3 log₁₀ (i.e., 99%-99.9%) reduction of bacteria colony count, as the host immune system response can generally overcome any remaining bacteria.

While the invention can be employed for killing or debilitating various pathogenic microorganisms, it can be used to advantage in treating H. pylori infections of the gastrointestinal system and other ailments where antibiotics are used with an increased risk of creating resistant stains of bacteria. By way of illustrative example, the present invention is described in the treatment of H. pylori infections within the stomach. It should be understood, however, that the invention is not limited to specific devices or procedures described herein. It is understood that the general principles taught can be used in other organs and parts of the body and on other organisms. Further, various devices and procedures are described for producing sufficient light, with an understanding that someone of ordinary skill in the art will not be limited to these examples.

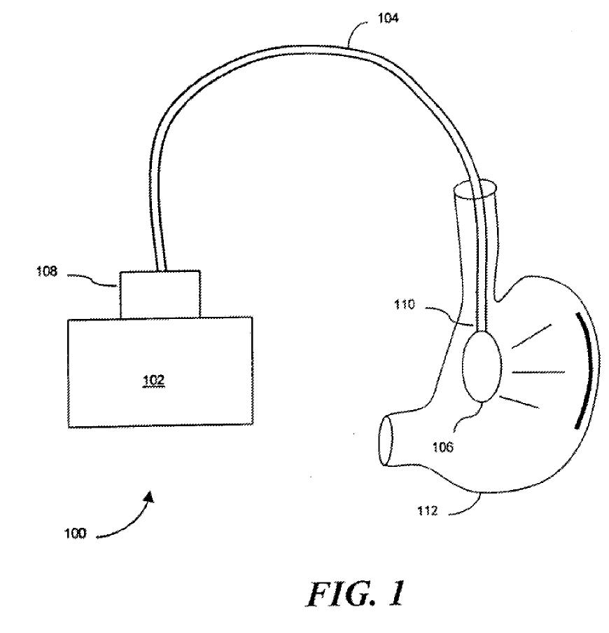

Referring now to FIG. 1, one embodiment of a light treating device 100 includes a light source 102 provided external to a patient's body, a light guide 104 for directing at least a portion of light emitted from the light source into the patient's body, and a delivery element 106 for delivering at least a portion of the directed light to a target location 112 within the patient's body. The light source 102 emits electromagnetic radiation having preferred wavelengths in the visible spectrum and is optically coupled to a proximal end 108 of the light guide 104. Generally, the light source 102 provides sufficient power at the preferential wavelengths for treating a microorganism, such as H. pylori. The light guide further includes a distal end 110 dimensioned for insertion into the patient's body.

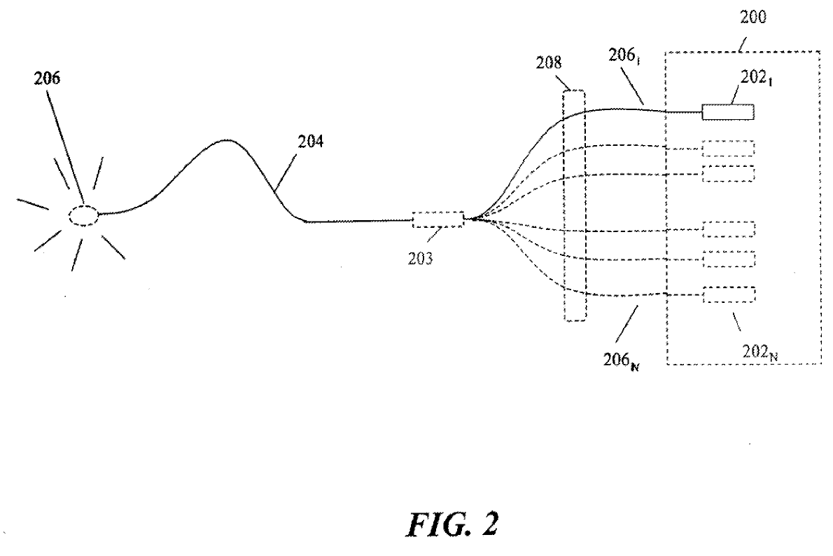

In another embodiment, referring to FIG. 2, an external source includes an array 200 of light sources (e.g., light sources 202₁ . . . 202_{N}, generally referred to below as light source 202). In the case of an external array 200, the total light emission from the array can be combined, for example, by an adapter/combiner 203. The adapter can include one or more reflectors (e.g., mirrors), lenses, prisms, and/or fiber optic strands that function individually, or in combination to couple a substantial amount of light energy into a light guide 204. This light guide 204 then directs the coupled light to a location within a patient's body.

In one embodiment, each light source 202 of the array 200 can include a laser diode (e.g., Nichia Corp. brand diodes), having a primary emission wavelength of approximately 405 nm. The laser diode package typically includes a fiber optic pigtail. Thus, light from the multiple light sources 202 can then be coupled into a single light guide, or fiber optic bundle 208 by combining the pigtails 206₁ . . . 206_{N}, generally 206 using an N-to-1 splicer, and/or a lens. The emission from this bundle 208 can then be coupled into a light guide consisting of a single fiber or multiple fiber bundle. In one embodiment, a removable/replaceable light guide 204 can be sterilized before insertion into a patient's body (e.g., being passed through a gastroscope). Additionally, the insertable portion of the removable light guide 204 can be detached from the light source 202 for replacement, for example, with another light guide sized and/or otherwise configured for a different application.

In one embodiment of this invention, the light sources 202 include argon ion lasers, each approximately tuned to a 457 nm emission line. In another embodiment, the light sources 202 include laser diodes (e.g., Melles Griot brand diodes), operating at approximately 457 nm. In yet another embodiment, the light sources 202 include HeCd lasers, operating at approximately 442 nm.

Thus, light from the external source 102, 202 is delivered through a light guide 104, 204 into the stomach sufficiently undiminished to effect bacterial eradication. In addition, the delivery means 104, 204 and the light delivery element 106, 206 are small enough in diameter to pass either through the mouth and esophagus and into the stomach, or through a working channel of a standard flexible endoscope previously positioned with its distal end in the stomach. Once the guide has transmitted the light to the stomach, this invention comprises numerous approaches for diffusing the light to provide complete illumination of the inner surface of the stomach.

In another embodiment, the light source 102, 202 can be a light emitting diode (LED), such as high output blue-violet devices manufactured by Nichia Corp., of Tokushima, Japan, or LED devices manufactured by CREE, of Durham, N.C.; a lamp, such as an incandescent lamp, a florescent lamp, or an arc lamps manufactured by Hamamatsu Corp., of Hamamatsu City, Japan.

The light guide 104, 204 transfers at least a portion of emitted light from the light source 102 to a location within a patient's body. In various embodiments, the light guide 104 is flexible thereby facilitating insertion and removal, and manipulation within the patient's body. For example, the light guide 104 can be a fiber optic cable, such as a glass and/or plastic fiber optic cable including a core, a cladding, and optionally, a jacket. In some embodiments, the light guide 104 includes a fiber optic bundle including more than one fiber optic cable. Such a bundle generally allows for a greater transfer of light than a single fiber, and also provides some flexibility in directing light at the distal end 110. Further, in some embodiments, the light guide 104 includes a hollow tube containing a gas and/or liquid therein. Transfer of visible light occurs through the gas and/or liquid. Some examples of gas include air, nitrogen, and argon. Some examples of liquid include water, and fluorocarbons.

In one exemplary application, a light treating device 100 for illuminating the inside of a patient's stomach 112 includes a transluminal light guide 104 having a length of at least approximately 150 centimeters (cm) to extend from the inside of the stomach 112 to the light source 102 located outside the patient's body. The outside diameter of the distal end 105 and the light delivery element 106 can be sized (e.g., 2-3 mm) to fit within a lumen, such as a provided by a surgical instrument (e.g., a catheter, an endoscope, or a gastroscope). Alternatively, the outside diameter of the distal end 105 can be provided with a larger diameter (e.g., 8-12 mm) for insertion into the stomach 112 for use within a larger catheter, for insertion without a catheter through the mouth and esophagus.

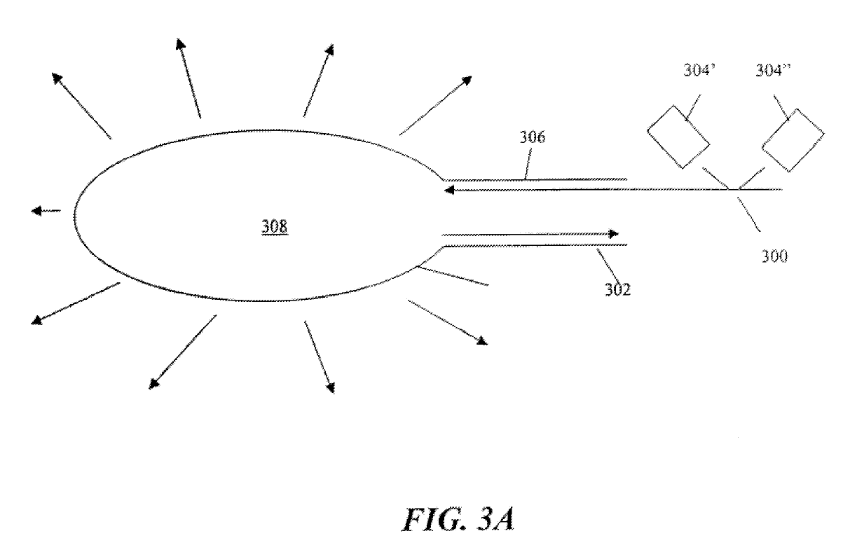

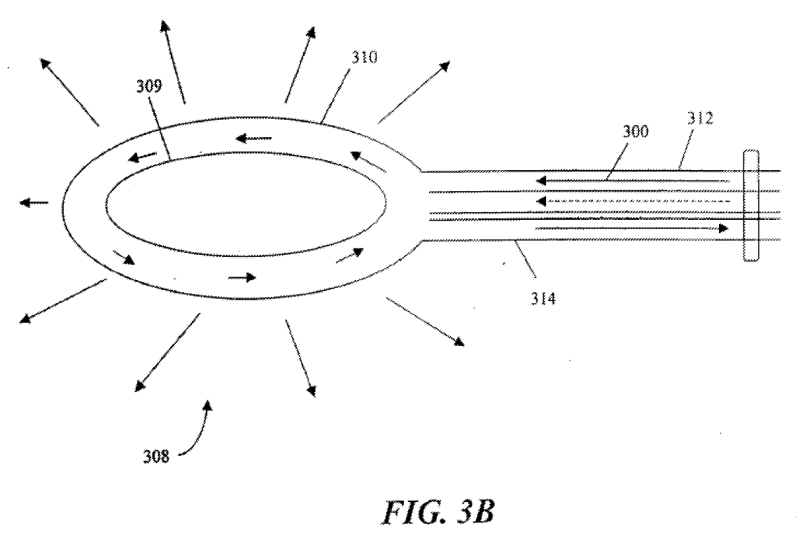

Referring to FIG. 3A, in one embodiment, light is conducted through a flowing biocompatible liquid (e.g., water) stream 300 that is conveyed via a hollow tube 302. The external light source 304′, 304″, generally 304, is coupled into the fluid 300 carried in the tube 302. The fluid 300 is selected such that t does not substantially absorb the light wavelengths of interest. Rather, the light reflects and refracts through the fluid 300 and off the walls of the tube 302 so that the light is substantially delivered to the tube's distal end 306 in the stomach. At the distal end 306 of the guide, the fluid is directed via a delivery element 308 to the stomach lining, thereby delivering light directly to the inner surface of the tissue. In one embodiment, the delivery element 308 is an expandable structure, such as a balloon. Thus, the liquid 300 can be delivered to the balloon 308 at a flow rate and pressure selected to control the inflation of the balloon, in turn, controlling inflation of the stomach. Such inflation tends to smooth out any naturally occurring folds and wrinkles of the stomach. In this embodiment, the light source 304 can be coupled into the light tube 302 through a wall of the tube.

In another embodiment, referring to FIG. 3B, the delivery element includes a double-walled delivery element 308. The fluid 300 is injected through a first tube 312 into the delivery element 308, and exits at a second tube 314. The fluid 300 flows between the inner 309 and outer walls 310, thereby confining the fluid 300 along the surface of the delivery element. The double-walled element 308 increases the operating efficiency by reducing the volume of fluid 300 necessary to cover a given surface area.

In some embodiments, a flexible fiber optic device is provided, which includes components for producing high intensity light and, optionally including an inflatable balloon surrounding the distal tip of the fiber optic device where the balloon acts as a diffuser, centering device, and an expander for the walls of the body cavity.

There is considerable information available in the field for preparation and application of flexible fiber optic guides in medical practice. Referring again to FIG. 1, in some embodiments, adapters 102 are optically coupled between the light source 102 and the light guide 104. The adapters 102 include standard optical connectors and/or splices for coupling light into a fiber or fiber bundle light guide 204. In addition, for the array 200 of FIG. 2, a multi-strand connector or N-to-1 coupler or one or more lenses can be used to funnel the light to the light guide 204. The fiber or fiber bundle 104, 204 is selected of a material so that near complete transmission of the light is accomplished and it passes substantially undiminished into the stomach. The guide 104, 204 is small enough to pass into the stomach from the mouth, or small enough to pass through the working channel of a standard medical endoscope. The distal end of the fiber is extended into the stomach and the light diffused or distributed for broad illumination of the stomach. If the diffuser/power combination isn't sufficient to provide a sufficient light “dose” to the entire inner surface of the stomach at one time, the medical practitioner can move the guide 104, 204 thereby “sweeping” the light delivery element 106, 206 until the entire stomach is treated. The sweeping action can include translation and/or rotation of the delivery element 106, 206.

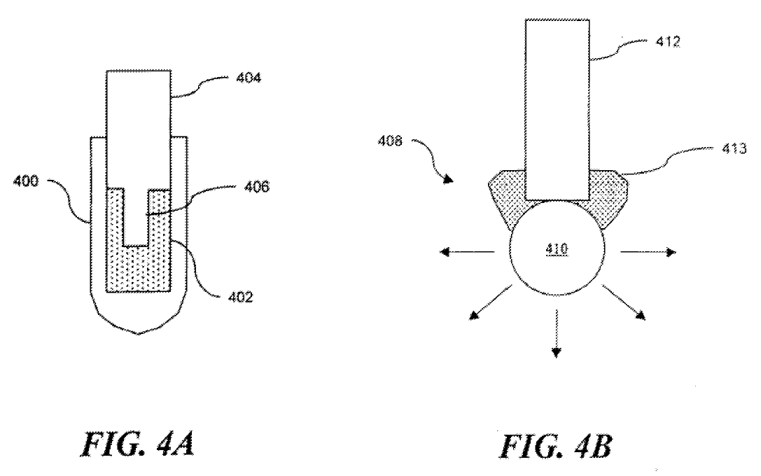

When treating a gastric infection of H. pylori with a light guide 104, 204, it is necessary to spread out the light broadly on the surface of all or part of the stomach. In one embodiment shown in FIG. 4A, a diffusing tip 400 is used for this purpose. The diffusing tip 400 includes section of refractive index-matching material with a dispersing medium, typically suspended reflective particles 402, is attached to a distal end of the light guide 404. The region of the light guide 404 within the diffuser 400 has its cladding removed to allow the light to pass through the wall of the fiber(s) 406 and into the diffusing medium 402 to produce a diffuse beam. The diffusing medium 402 can be contained within a container, such as a balloon. There are many materials, types and geometries of diffusing tips known to one skilled in the art. Most diffusers create a cylindrical pattern of illumination around the end of the guide. Some diffusers are sized slightly larger than the diameter of the light guide 104, 204.

According to yet another embodiment, a delivery element 408 can include a variety of lens shapes, such as a spherical dispersing bead 410 shown in FIG. 4B. The dispersing bead 410 is employed to spread out the light at the distal end of a light guide 412. For example, the spherical bead 410 can be made of epoxy or fused silica formed with a fusion splicer 517 on the guide distal end. The spherical bead 410 disperses the light rays in a nearly complete spherical pattern. To assure complete light coverage over an entire region of interest, it again may be necessary to move or sweep the light delivery element 106, 206 within the stomach. There are many materials, types and specific geometries of spherical disbursing beads 410 known to one skilled in the art.

Some examples of light sources located external to a patient's body have been described above. These light sources have many advantages, including the use of electrical (e.g., A.C.) power and the ability to illuminate for an indefinite period of time. Further, since there are substantially no limits to the size and capacity of a power source, the typical external light source can be relatively powerful, filtered as desired, and readily available for other illuminating applications, both medical and non-medical.

However, there are also a number of advantages of generating the light for treatment directly in the stomach or other area of interest. At least one advantage includes patient convenience. Light internally generated can eliminate the need for an endoscope. Internal light sources can include a tube, or more generally, a tether that is substantially smaller than an endoscope leading from within a patient's body to the outside. Even more beneficial, in some applications, no external connection is required at all.

Another advantage relates to the duration of treatment. Use of an endoscope can only be done by a highly trained specialty physician and for a limited amount of time. An internally generated light can allow for treatment by less highly trained individuals thereby reducing and/or eliminating the need for expensive specialists.

Another method to supply light to the stomach or other organ to treat H. pylori infection is by use of tethered or self-powered lamps. There are numerous types of lights that can be delivered inside a patient's body (e.g., to the stomach) through a natural body lumen (e.g., the esophagus). These lights include a camera flash or strobe technology. Flash and/or strobe lamps are generally powered by low voltage direct current (DC) energy sources, such as batteries charging a capacitor, that when triggered, “fire” a flash lamp, such as a xenon or other gas-filled miniature arc lamp to produce high intensity light pulses. Rapid, short pulses of intense light in the wavelength of interest have been known to have enhanced effectiveness over continuous wave (CW) light delivery in certain applications. Pulsed light providing a time averaged delivery of energy comparable to a CW source, can provide peak intensities that are substantially greater than the CW peak energy. Further, short duration pulses of high intensity result in non-linear effects, some of which, while not fully understood, appear to enhance certain biological effects. These advantages will be understood to be applicable to all of the embodiments described in this application, including the delivery of light using external light sources, described earlier in this application.

Another advantage of a low-voltage charging system, is the inherent safety to the patient. A low voltage system has essentially no risk of a damaging electrical shock. The entire system of power supply, batteries and flash are all small enough to be encapsulated and swallowed or advanced into the stomach of the patient. This technology has been developed for the photography industry, and is very small and compact.

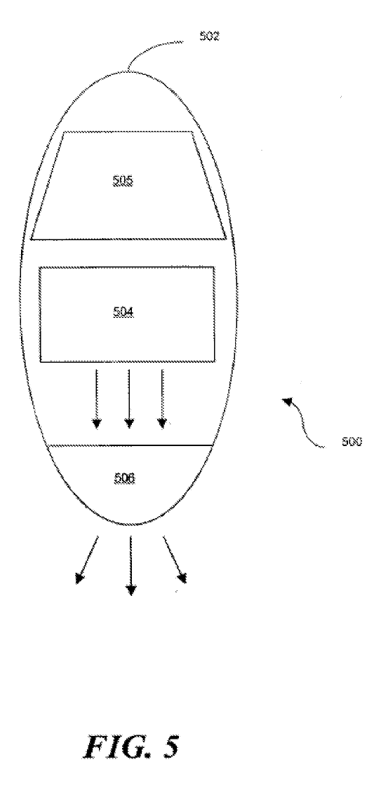

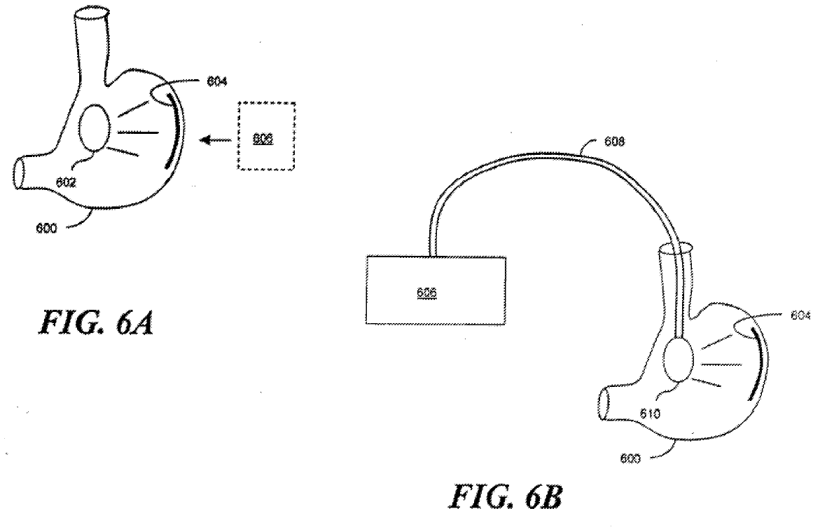

Referring to FIG. 5, a swallowable internal light treatment device 500, includes a housing 502 sized and shaped to facilitate insertion into a body, e.g., swallowing. The housing 502 includes an internal light source 504 and an on-board energy source 505. The energy source 505 energizes the light source 504, resulting in the emission of electromagnetic radiation substantially within the visible region. In some embodiments, the device 500 includes a light delivery element 506 for delivering light to a target location within a patient's body. FIG. 6A illustrates an untethered device 500 dimensioned and shaped for insertion into a patient's stomach 600 for delivering light 602 to a target location 604.

In another embodiment, the device 500 includes a tether to an external power source 606 as shown in FIG. 6B. A tethered light treating device 701 includes an external energy source 606, coupled via a tether 608 to a light source 610 dimensioned for insertion within a patient's body (e.g., within the stomach 600). The energy source 606 can include an electrical energy source, such as a battery, or power supply, or an optical energy source providing light through a light guide tether 608. The energy (electrical, optical) is received by the light source 610 which converts the energy into visible light at the preferred wavelengths. For example, the light source 610 includes laser diodes, LEDs, lamps that can be powered by electricity, or the light source 610 includes a light-emitting material that radiates (e.g., florescence) when illuminated by the energy source.

Referring again to FIG. 6A, in another embodiment, an untethered light source 500 is powered by an external energy source 606. The external energy source 606 provides energy in a transcorporeal manner to the light source 500, thereby energizing the light source 500 to emit the desired visible light. In one embodiment, the external energy source 606 includes a transformer for coupling electrical energy to the light source 500. In other embodiments, the external energy source 606 includes beamed electromagnetic energy that causes microwave induced emissions within the light source 500. For example, incident electromagnetic energy can be captured, rectified, and converted into usable electrical energy within the light source.

Another method to supply light directly in the stomach is by the use of a single-use incandescent flash bulb. A small device housing a fine filament and an igniter are swallowed or advanced by the clinician into the patient's stomach. Once in position, the magnesium filament flash lamp is fired to produce an intense flash. Although this is a single flash, it's output from the combustion of the filament is high enough to supply the total number of Joules required for the therapy. Magnesium, for example, produces a very intense white flash. If necessary, appropriate filtering can be done around the filament to tailor the light wavelength closer to the absorption of the H. pylori. Other materials can be used for the filament if necessary to increase the power delivered, get more light into the primary band of interest, or make it easier to ignite the filament. In one embodiment, the intense flash creates heat that is cooled to avoid damage to the stomach or other internal tissue. Cooling can be done by circulating fluid or by other means known to those skilled in the art.

Another method to supply light directly in the stomach is by the use of a miniature fluorescent or arc lamp. These lamps are higher voltages than the flash lamps described above, so additional electrical insulation and care are used to avoid the risk of electrical shock to the patient or clinician. These lamps are typically low current, low heat lamps so the need for thermal cooling is diminished. One advantage of these types of lamps is that much of their power can be designed to transmit light in the blue/violet wavelengths, the light of most effectiveness for eradication of H. pylori or other bacteria killed by endogenous or administered porphyrins.

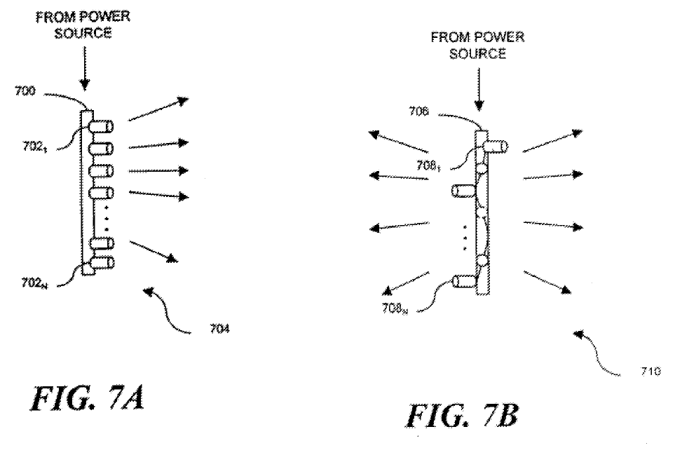

Another method to supply light directly in the stomach is by the use of miniature Light Emitting Diodes (LED's) or laser diodes. These semi-conductor devices are small, emit light in a very narrow wavelength, are very energy efficient, and generally create only a small amount of waste heat. Each individual device is quite small, and delivers only a fraction of the total illumination necessary, however, due to their small size and low cost, many devices can be grouped together for a more powerful delivery device. FIGS. 7A and 7B illustrate arrays 700, 706 of LEDs including a linear array 702₁ . . . 702_{N}, generally 702, and a helical array 708₁ . . . 708_{N}, generally 708. For example, a single blue LED may emit only about 10 milliwatts of light at a wavelength of about 405 nm, but these devices are small enough that many of these could be assembled at the distal end of a catheter to deliver sufficient light for bacterial eradication within the stomach or other location with a patient's body. Although these devices 702, 708 are power efficient, and do not create a lot of excess heat, it may be necessary to actively cool them to avoid the potential of burning the patient or substantially decreasing the illumination life of the diodes. Once in the stomach, the LED array 704, 710 can be moved or rotated through the area of infection, or can be inserted into the stomach within a balloon, which when inflated keeps the array at a known distance from the stomach wall. Liquid can be circulated through the balloon to assist in the cooling of the device.

Another method to supply light directly in the stomach is by the use of electron beam excitation, one form of which is also known as Cerenkov radiation. When certain materials are struck by an electron beam, they emit photons. If the material is selected to emit photons in the wavelength around 405 nm, this method can be employed to eradicate H. pylori. For applications in which the beam can not be directed through the body directly, it can be directed into the stomach via a series of reflectors and/or a tube.

A delivery element 106, 206 delivers light to a target area. The target area may be confined to a localized region, whereby a focused beam delivers light to the localized region. In other applications, the target area may include substantially all portions of the stomach. Thus, a suitable delivery element 106, 206 disperses a beam to deliver light to a larger region. For applications in which it is impractical to generate a single light beam to cover the entire target area, the delivery element 106, 206 can be moved as necessary.

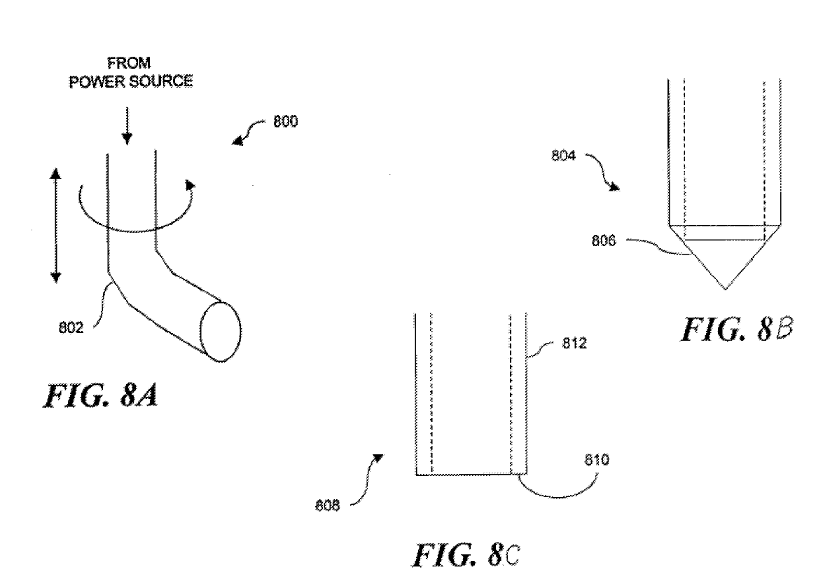

For example, referring to FIG. 8A, a delivery element 800 includes an angled tip 802 that can be rotated and/or translated up and down, as illustrated, to “sweep” the light through a path over the entire stomach.

In another embodiment, referring to FIG. 8B, a delivery element 804 includes a tapered end 562. The pattern of light projected from the tapered end 562 is conical or a similar shape. The taper near the guide tip refracts rays forward and outward into widely divergent beam. The projection of this beam is a circle on a flat surface. This type of tip is commonly used during PhotoDynamic Therapy (PDT) for various treatments for cancer, pre-cancerous conditions like Barrett's esophagitis, etc. To assure complete light coverage over the entire region of infection, it may be necessary to move the delivery element 804 with tapered tip 562 within the stomach. There are many materials, types and specific geometries of tapered tips known to one skilled in the art.

In another embodiment, referring to FIG. 8C, a delivery element 808 includes a flat or convex polished fiber end 810. In application, the fiber end 810 of the light guide can be positioned at the cardiac orifice, the entrance to the stomach from the esophagus. As this light guide 812 provides the light to the entrance of the stomach, the light is diffused and distributed over the entire stomach inner surface. Diffusion of the light can be accomplished in a number of ways. For example, the stomach can be filled with a light diffusing liquid. The light rays will diffuse throughout the liquid and will be absorbed when they reach the surface of the stomach making the stomach the equivalent of an integrating sphere.

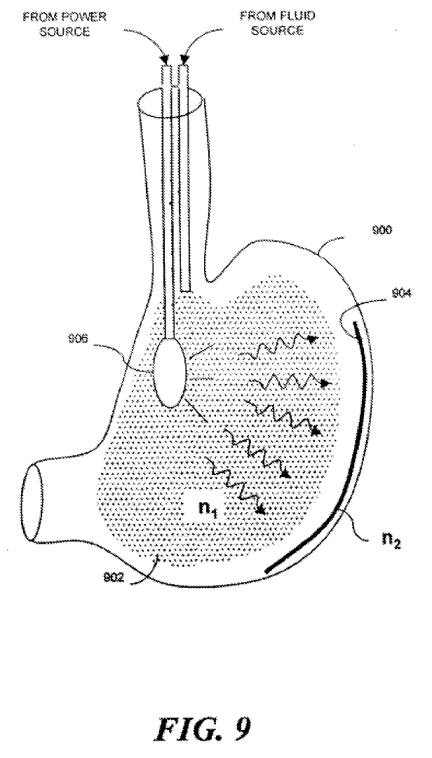

In another embodiment referring to FIG. 9, the stomach 900 is filled with a substantially transparent fluid 902 having a refractive index (n₁) that is higher than a refractive index associated with the mucus lining of the stomach (n₂) 904. Internal reflection at the interface between the fluid 902 and the mucus lining 904 occur trapping those light rays within the fluid 902, provided by a delivery element 906, that are incident upon the mucus lining 904 at a reflective angle less than a critical angle determined by the two refractive indexes. The light rays will then be distributed substantially uniformly throughout the stomach 900 and those rays that exceed the critical angle will then penetrate the mucus layer and reach the infected regions of the stomach 900.

In another embodiment, the lining of the stomach 904 is first coated with a transparent fluid of selected (low) refractive index (n₂) the stomach is then filled with a transparent fluid 902 of higher refractive index (n₁) than the first layer. For the same reasons described above, the light rays will be distributed substantially uniformly throughout the stomach 900 and those rays that exceed the critical angle will then penetrate the mucus layer 904 and reach the infected regions of the stomach 900.

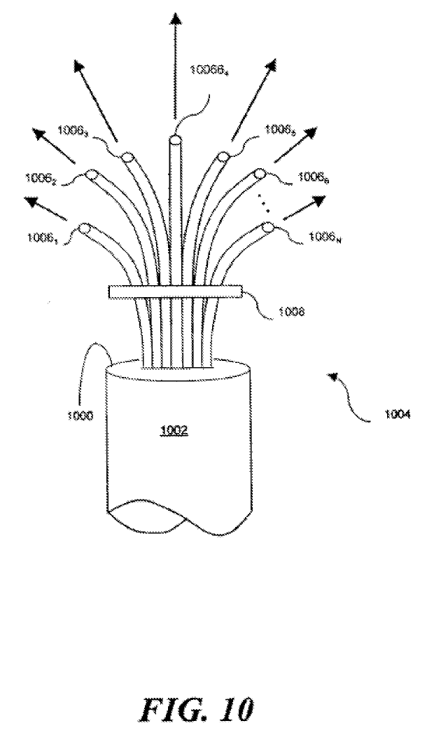

Referring to FIG. 10, another way to spread out the light at the distal end 1000 of the guide 1002 is to employ a fiber optic “fountain” 1004. The distal end 1000 of a multiple fiber bundle is separated into individual fibers (e.g., fibers 1006₁ . . . 1006_{N}, referred to collectively as fibers 1006) to “spray” light in all directions. The fibers 1006 can be supported by a support element 1008 to substantially hold the fibers in a disruptive arrangement. The ends of the fibers 1006 can be further treated to remove the cladding or by adding diffusers (not shown) to increase the area illuminated. This “fountain” 1004 or “brush” can be set into motion to further distribute the light or can be swept along the inside lining of the stomach to make contact and effectively “paint” the surface with light, by means of frustrated internal reflection (i.e., index matching). (This geometry is similar to novelty shop fiber optic trees).

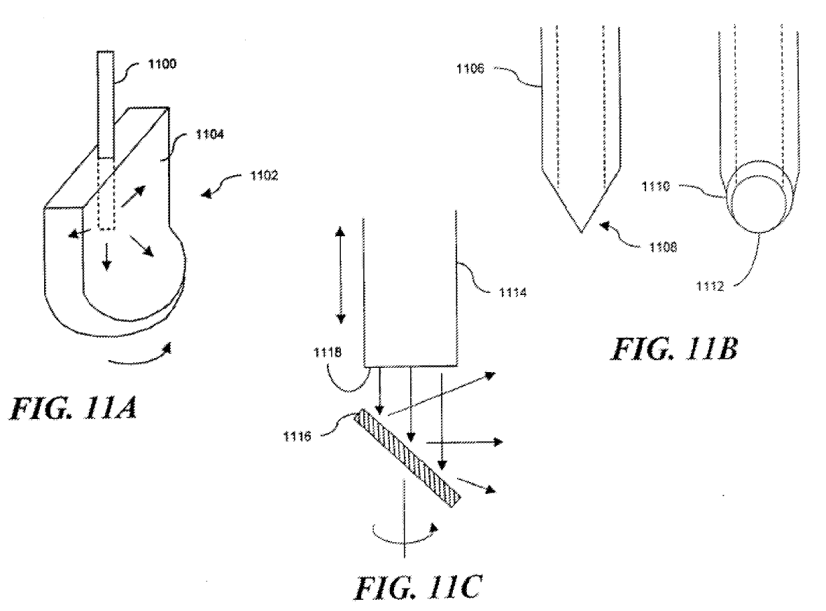

In another embodiment, referring to FIG. 11A, coupled light can be delivered at the distal end of a light guide 1100 by employing a flexible paddle shaped tip 1102. In this configuration, the light guide 1100 terminates in a flexible light transmitting “paddle” 1102 that is passed along the inner surface of the stomach for direct contact delivery of light. The paddle 1102 can be a separate flexible part, for example, it can be made from clear silicone rubber. A silicone paddle 1102 flexes and adheres via surface tension to the inner wall of the stomach as it is swept along the surface. The material of the paddle transmits the light from the guide 1100 to the edge or surface of the paddle 1104 in contact with the stomach wall. Light is thus transferred from the paddle to the stomach wall as a consequence of a near match in refractive index between the two. The flexible paddle 1102 can be rolled or coiled-up for introduction through the endoscope or esophagus. Once in place, the paddle 1102 would automatically unfurl or could be unfurled by the practitioner using a release mechanism.

In another embodiment, referring to FIG. 11B, the distal end of a light guide 1106 is formed in a wedge shaped end 1108. The distal end 1106 of the light guide can be polished on either side of a center line forming the wedge shaped end 1108. The portion of the light guide at the wedge shaped region 1108 has its cladding 1110 removed resulting in lateral “windows” 1112, which direct light out through the tip and sides of the wedge shaped end 1108. The light guide 1106 can be rotated and/or moved transversely to achieve complete illumination of the stomach interior.

In yet another embodiment, referring to FIG. 11C, light can be spread out at the distal end of the light guide 1114 by employing a rotating and/or oscillating mirror 1116, lens, or prism. Rotating mirrors 1116 are known in the medical field, particularly for use in intraluminal ultrasound, where a rotating sound reflecting and receiving mirror is positioned at the distal tip of a coronary catheter to provide ultrasound images from the lateral arterial wall. In the application for H. pylori treatment, the light reflecting mirror 1116 is positioned at the distal end 1118 of the light guide and as the mirror 1116 rotates, it “bathes” the interior of the stomach with light. The mirror is selected to be a good reflector of substantially all the light arriving through the light guide 1114. Many methods of creating and rotating the mirror are known to those skilled in the art. In order to completely treat the entire stomach inner surface it may be necessary to move the guide longitudinally while rotating the mirror.

In still another embodiment a delivery element includes a balloon to help distribute light completely over the region of interest. The balloon can be constructed with a partially reflecting inner or outer surface, such as a “half-silvered” surface. Such a partially reflecting surface results in multiple internal reflections from a light source provided inside the balloon. After multiple reflections, a portion of the light will find its way out of the unreflective spaces in the balloon, thereby insuring an even distribution of light. Using a balloon inside an organ for complete light dispersion is known to those skilled in the art as a complete integrating sphere.

Gastric balloons are well known by those skilled in the art for many purposes. Balloons can be employed in this therapy for a number of advantageous reasons. For those applications where the organ geometry is not simple, yet a uniform dose of light is desired, a special modification of the partially reflecting balloon can be employed, whereby the transmittance of the balloon increases with the balloon inflation diameter. With this modification, the physician can adjust the delivered dose automatically by inflating the balloon to fit any portion of the organ cavity. In this way, those portions of the organ that have a larger diameter and are thus more distanced from the light delivery means that is centered within the balloon, will receive an equal dosage as compared with those portions of the organ that are of smaller diameter.

A balloon can be filled with a light scattering liquid medium, such as milk or reflecting particles, such as talc and/or titanium dioxide suspended within a fluid, such as water. In addition to stretching the stomach and serving as a light diffuser for complete and uniform illumination of the region surrounding the balloon, the liquid also serves to absorb waste-heat that can be produced by the light source. The balloon can be made from an elastic material such as latex, silicone rubber or polyurethane. The balloon can also be made from a non-elastic material that unfolds or unrolls as it is inflated, filling the stomach. In this example the unfolding balloon can actually be more of an inflatable bag than a stretching balloon. Both the inelastic and elastic structures are known as balloons to those skilled in the art. The non-elastic balloons can be made from polyethylene, polypropylene, nylon, polyvinyl chloride, polyurethane (of a less elasticity than the material used for the expandable balloon described above). In all cases, the balloon material is sufficient to transmit the light wavelength of interest to allow for effective illumination and treatment of the bacteria.

The light guide can be inserted into the stomach through the shaft of a balloon catheter and the assembly inserted in the stomach. Alternatively, the balloon catheter can be placed in the stomach and the light guide subsequently advanced into the catheter. When the balloon is inflated in a manner so that it fills and slightly distends the stomach, the light guide is centered in the stomach. Alternatively, the balloon can be smaller than the entire stomach and fulfill the function of a bumper for safety and to keep the light guide away from the wall of the stomach. The balloon can be registered against the stomach entrance or stomach exit or within the stomach, to center or provide a path for the light guide.

In some embodiments, a mechanical positioning element facilitates location and/or movement of the delivery element 106, 206. For example, a plug can be provided on the outside of a flexible endoscope or on a device inserted directly through the esophagus and into the stomach. The plug, or collar, allows an operator to register the tip location of the light guide 104, 204 against the cardiac orifice. In addition, the plug supports the light guide 104, 204 or associated tether 608. For example, a small light source 610 swallowed by the patient with the associated tether 608 (e.g., wires and optional cooling mechanism) extending out of the patient through the esophagus. The plug is provided around the tether 608 temporarily lodging it in the lower esophagus or upper stomach for support or anchoring. The plug then provides a stationary or sliding support for the delivery element 106, 206, 610 so that it can be moved in and out or rotated to project the light over the entire stomach.

Alternatively, or in addition, the position of the delivery element 106, 206, 610 can be directed by providing a ferromagnetic section thereon. The ferromagnetic section can thus be manipulated in a transcorporeal manner using an external magnetic.

Gas filled balloons can also be used. In a like manner to the liquid filled balloon, the stomach is stretched in an attempt to flatten and expose ridges and alveoli. The same materials described for the liquid filled balloon can be used for gas filled balloons. Advantages of filling the balloon with gas include quicker filling and deflating of the balloon, no absorption of the light energy by the liquid, and the gas filled balloon can be more comfortable for the awake patient.

In some applications, such as treatment of the stomach, distortion of a location within the patient's body is beneficial. For example, it is beneficial to illuminate the entire stomach, as the bacteria may live anywhere in the stomach and may be living in colonies not connected with other areas of infection. Although certain areas of the stomach are more prone to infection, it is not feasible to determine in advance or at the time of treatment the specific areas of infection. Therefore, it is a prime consideration of this invention to treat, in the most effective and simplest way, the entire stomach. Distending the stomach smoothes out the folds and other features of the stomach, decreasing the chance that a portion of the stomach will be in shadow from the light source. In addition, distending the stomach gives more space for the light guide or other light source to maneuver in the stomach and makes visualization with an endoscope easier. Further, expansion of the stomach exposes ridges and glands or crypts, the small pores in the wall of the gastric endothelial lining where mucous and acid are produced. H. pylori may live in these glands, beneath the mucous layer. Additionally, inflation of the stomach aids in thinning the mucous layer, stretching out the glands as well as the larger features like the rugae, all improving the success of illumination of the bacteria.

Inflation can be accomplished using a gas, a balloon (transparent to the light source used) and/or a liquid. Any liquid or gas used is generally biocompatible and safe for use in the stomach. One example of a liquid would be to fill the stomach with milk or other liquid with a good suspended light scattering medium such as Pepto Bismol® or other antacid liquid medication. These light scattering liquids help to assure that the entire stomach surface is illuminated. In addition, the fluid also serves to absorb any waste-heat generated by the light source, as hundreds of Joules of energy can be delivered to effect complete eradication treatment.

Another example of an inflation fluid is the transparent liquid with higher refractive index, described earlier as a means of rendering the stomach as an integrating sphere. Another example of inflation method would be swallowing of a gas producing tablet or capsule that releases the gas in contact with gastric juices. The capsule could be calibrated or selected to provide the optimal amount of gas for distension of the stomach without pain or excess gas.

One of the challenges of this therapy is to assure treatment by illumination of all portions of the gastric mucosa. One additional means for assuring complete light “coverage” would be to coat the inner walls of the stomach with a liquid containing light dispersing particles in the medium. The liquid would ideally be low enough in viscosity and high enough in adhesion to allow for a small volume of liquid to completely coat or cover the stomach. The liquid would ideally be able to mix or adhere to the mucous layer coating the entire stomach, and remain in intimate contact with the mucous or endothelial layer for sufficient time for complete illumination therapy to be completed.

In another embodiment, a mechanical support, such as a retractable fine wire or plastic filament cage can be inserted into the stomach. Once in the stomach, the cage expands until it gently pushes out the inside wall of the stomach. In addition, the cage can have a smaller diameter section near the top of the stomach or somewhere along the long axis of the cage to provide a stationary or sliding support for catheter or other portion of the light guide or illumination device.

Alternatively, or in addition, other advantages can be obtained by deflating the stomach. These advantages include making the surface to be treated smaller, as the treatment will require a minimum amount of energy (Joules) delivered to each square centimeter of surface area. The smaller the surface area to be treated, the lower the amount of power required for the treatment. In addition, deflation offers a means of equalizing the distance between the end of the light delivery means and the infected tissue. In this way, delivered dosage can be equalized, as light disperses from a point source in an inverse squared function: the closer the stomach wall is to the light source, the more illumination energy provided to an area. Another advantage of deflation is that the stomach wall can be stretched selectively over a substrate of particular shape. Another advantage of deflating the stomach is that when it is deflated or flattened against the light source, for example, against the flexible paddle tip described above, it may be easier to guide or center the light source or guide in the narrower space formed by the deflated stomach.

A light emitting material, such as a phosphorescent material can be activated by an energy source, such as a bright light, prior to insertion into a patient's body and caused to emit, during and for some time after removal of the activating energy source, electromagnetic radiation having wavelengths in the visible spectrum. The light-emitting material selected to be non-harmful to a patient when placed therein, and generally non-harmful to body tissues when placed in contact therewith for a limited duration.

In one embodiment, a phosphorescent material (“glowing fluid”) is prepared as a liquid for insertion into a patient's body. The liquid can be inserted through an artificial lumen, such as a catheter, or through a naturally occurring lumen, such as the esophagus. Thus, the glowing fluid can be ingested, thereby coating the stomach lining and delivering light energy of an appropriate wavelength to the stomach lining for eradication of pathogenic microorganisms, such as H. pylori bacteria. In this manner, the glowing fluid is placed in close proximity to the bacteria, so that the light intensity is not diminished (or spread) significantly as can occur when light radiates across a distance. Preferably, for treatment of the stomach, the glowing fluid is selected to enhance coating of substantially the entire stomach, thereby insuring irradiation of substantially all locations in which the bacteria may reside. Thus, coating of the stomach in this manner overcomes the difficulty of illuminating within and around the folds, pores, and textures of the stomach lining.

The light treating fluid can be washed away by normal fluid action within the stomach, by mechanically removing the fluid, and/or by ingestion of other liquids to dissolve the glowing liquid and/or speed in washing it away. If necessary, repeat ingestion or continuous pumping of the glowing liquid can be accomplished to deliver the necessary dosage of light treatment.

In another embodiment, a light-emitting material includes a chemiluminescent material. Chemiluminescence is a chemical reaction within a material that emits light. Generally, referring to FIG. 3A, the material includes at least two chemicals 1200, 1202 in liquid form are mixed together whereby the resulting chemical reaction 1204 emits electromagnetic radiation having wavelengths in the visible spectrum. Further, the material emits light for certain duration of time. The chemicals 1200, 1202 can contain both a dye or dyes that create the specific wavelength(s) of light 1206, and an energy releasing reaction species, that provides the energy required to “pump” the dye molecules to a higher energy state. When the dye molecule naturally relaxes from it's higher energy state, photons of a specific wavelength is released.

Chemiluminescence is well known to those skilled in the art, and it is embodied or described in many products, scientific articles and patents. The proper selection of the chemicals 1200, 1202 can provide certain light 1206 of a specific wavelength peak, or by combining multiple chemicals 1200, 1202 with different dyes, light 1206 of multiple peaks can be delivered. In addition, the chemicals 1200, 1202 can be selected to supply an energetic reaction providing a relatively rapid release of radiant energy, or, alternatively, the chemicals 1200, 1202 can be selected to supply a less energetic reaction providing a longer, slower release of radiant energy. Thus, to provide a low light intensity for a long time, the chemicals 1200, 1202 are selected for a slow reaction rate. The total number of photons delivered generally depends on the energy produced by the reaction 1204, the efficiency of the reaction 1204 in exciting the dye to it's higher energy state, and the efficiency in the excited dye molecules returning to their lower energy state.

One example of a chemiluminescent material include a children's party toy, including a small liquid containing breakable vial sealed within a liquid filled plastic tube. By squeezing or bending the outer plastic tube, the inner breakable vial is broken, releasing the vial's liquid to mix with the plastic tube's liquid. The resulting reaction releases a low illumination level for 12-24 hours. Examples include products, such as the GLOWSTICK® manufactured by Omniglow Inc., of Springfield, Mass.

Another example of a chemiluminescent material includes a temporary airway landing light source. In this example, a similar configuration is used having a breakable vial within a tube. When the inner vial is broken, the resulting energetic reaction creates a relatively intense light 1206, but for a much shorter duration of time, e.g., 30-60 minutes. The brightness of the illumination and the duration of the light 1206 depend on first order chemical reaction kinetics. That is, heating up the chemicals 1200, 1202 makes the reaction rate faster. For example, a reaction is approximately twice as fast with a 10 degree centigrade increase in temperature.

Chemiluminescent chemicals 1200, 1202 can be used to provide the light 1206 for eradicating illumination of H pylori in the stomach or bacteria in other biological regions. As chemiluminescence is a general purpose technique for creating light 1206 at a specific location, it is understood that many other clinical treatments and techniques can be used by one skilled in the art of photobiology.

In one embodiment, treatment to kill and/or debilitate H. pylori in the stomach uses chemiluminescence, chemicals 1200, 1202 that produce the wavelengths of interest (typically near a peak of 405 nm) delivering a sufficient dose (i.e., having a sufficient total energy in Joules). For example, the two chemicals 1200, 1202 can include two chemical dyes, such as DPHA and BPEN, that when mixed with appropriate activators create visible light 1206 having an illumination peak at 438 nm with additional peaks at 454 and 486 nm respectively. The energy delivered by these chemicals 1200, 1202 provides a proper dosage for substantially eradicating (e.g., reducing by 99%) H. pylori bacteria within the stomach. Dosage levels, for example, determined through in vitro and animal testing with blue/violet light in the 405 nm range, are generally adequate to provide H. pylori eradication when delivered at an 30-100 Joules/cm².

In human stomachs, the chemicals 1200, 1202 can be delivered in many ways. One way is to put a balloon 1208 into the stomach as described above. Once the balloon 1208 is in position, the two chemicals 1200, 1202 can be mixed outside the body and injected into the balloon 1208, inflating the balloon 1208 to the desired degree. The chemicals 1200, 1202 are left in the balloon 1208 until the desired light dose has been delivered, and the chemical mixture 1210 (i.e., reaction products from the mixing of the chemicals 1200, 1202) is then withdrawn from the balloon. The balloon 317 is then withdrawn from the patient, completing the therapy.

Referring to FIG. 3B, a more efficient use of the chemiluminescent chemicals 1200, 1202 is to employ a double-walled vessel 1212, for example, a double-walled balloon 317′, for the internal distribution of the chemicals 1200, 1202. This is most effective because the external emission of light 1206 from highly concentrated chemiluminescent liquids 1200, 1202 occurs for the most part at the surface of the liquids and to a depth of only a few millimeters. Thus, the chemiluminescent chemicals 1200, 1202 are introduced into the lumen between the inner and outer balloon of a double-balloon configuration 317′. If additional light dose is necessary, a second mixture of chemicals 1200, 1202 can be used once the light 1206 from the first mixture is sufficiently exhausted. Alternately, the two chemicals 1200, 1202 can be mixed in small doses continuously outside the body, and pumped continuously through the balloon 317′ or through a transparent tube coiled in the stomach.

For embodiments in which the chemicals are sufficiently safe for ingestion by the patient, the patient can swallow the mixture, or a health care practitioner can deliver the mixture directly into the stomach through a tube advanced through the esophagus for that purpose. Another delivery mechanism includes “swallowable” capsules including the two or more chemicals (e.g., dye and activator) to be ingested, each chemical is separated from the other by a membrane or barrier. Just before swallowing the capsule, the membrane inside the capsule is broken by squeezing or twisting the capsule, thereby activating the chemiluminescent reaction. The activated pill is then swallowed by the patient. This process can be repeated as needed to deliver a full therapeutic dose of light, for example, to eradicate the H. pylori bacteria.

In another embodiment, the chemiluminescent material is administered using time release capsules. A time release capsule leverages the initial chemiluminescent light reaction, which is the most intense period of photon production. For a time release embodiment, refrigeration techniques can be employed to delay or slow the chemiluminescent reaction. Prior to insertion into a patient's body, the chemiluminescent reaction would later be initiated by the internal body temperature of the patient upon administering the capsules.

Alternatively, solvation methods can be used to dissolve reaction barriers between the reactants. Solvation can be hastened or retarded by adjusting the capsule temperature. Thus, a chemiluminescent reaction can be initiated by the patient's body temperature at the time that reaction is desired.

With any of the above capsule embodiments, additional capsules can be swallowed periodically as a preventative measure to minimize any chance of re-infection. Capsules can be designed to float. When such buoyant capsules are swallowed in combination with a liquid, such as water, they will float on, or near the surface of the liquid, thereby illuminating the top portion of the stomach. As the liquid drains from the stomach the capsule, continuing to illuminate the stomach, will then move downward providing complete light coverage of the rest of the stomach area. Further, passage of the capsule into the duodenum provides light coverage for that anatomical region.

In another embodiment, a chemiluminescent material is prepared directly within the patient. For example, a first material, such as a liquid is applied directly to the target location, e.g., the interior surface of the stomach tissue. The liquid can be applied endoscopically by dripping, painting, or spraying. Then a second material representing an activator is similarly applied to the same general area along the interior surface of the stomach. Light is produced upon the mixing of the two components, essentially at the surface of the tissue. Having a light intensity that is highly localized at the surface, where microorganisms, such as the H. pylori bacteria is high, further facilitates eradication of the microorganisms.

In another embodiment a sonoluminescent material is provided within a patient's body. The sonoluminescent material is activated through the application of sound waves (e.g., directed high intensity sound waves) to the sonoluminescent material. Sound energy activates the sonoluminescent material, for example, by creating cavitation in a liquid thereby resulting in the generation of electromagnetic radiation by the liquid. Preferably, the radiation includes wavelengths in the visible spectrum by the liquid. The light is created when the cavitation energy excites a chemical species to a higher energy state, enabling the releases of photons when it relaxes to the lower energy state. Using appropriate dyes within a liquid, such as water, the wavelength of light produced can be tailored. Thus, application of ultrasound energy to a sonoluminescent material can create sufficient light to treat pathogenic microorganism, such as H. pylori or other bacteria.

The acoustic and/or ultrasound source can be inserted within the patient's body together with the liquid, for example through an endoscope or catheter. Alternatively, the acoustic energy can be administered in a transcorporeal manner, as is commonly performed in the treatment of kidney stones (i.e., lithotripsy).

In another embodiment, microwaves and/or other electromagnetic waves are used to induced luminescence within a material. For example, one or more electromagnetic energy beams can be directed through body tissues and focused therein to a location within a patient's body, such as the stomach cavity. Techniques for focusing electromagnetic energy beneath a patient's skin are generally known, and employed, for example, in the radiation treatment of tumors. Prior to, or simultaneous with the radiation, a susceptor is provided within the patient's body. The susceptor is selected and incandesces upon illumination by the electromagnetic energy source.

In one embodiment, a susceptor, such as a dye is provided within the body of a patient. The dye can be provided directly within the patient, for example injected, or ingested into the stomach. Alternatively, the dye can be first placed within a container, such as a balloon, etc., the container then being inserted into the patient's body. The dye is activated by an external energy source, such as a microwave energy source, resulting in the dye emitting electromagnetic radiation. The dye in combination with the external energy source can be selected to produce light of a particular wavelength including wavelengths in the visible spectrum. In this manner, a substantial amount of light energy can be delivered to a remote location.

In another embodiment, combustion of incandescent materials, such as highly incandescent materials (e.g., magnesium) emit intense electromagnetic radiation over a broad range of wavelengths including visible spectrum (e.g., white light) when oxidized. One example of such a reaction includes disposable flash bulbs. Such combustible materials can be fed continuously to a suitably filtered and cooled reaction chamber that is introduced to the stomach via a catheter or endoscope. The resulting oxidation reaction can thus be maintained in a substantially continuous manner. Alternatively, a number of discrete oxidation reactions (“flashes”) can deliver a pulsed light source.

Another method to supply light directly in the stomach is by the use of radioactive decay of certain elements. Again, light is emitted by certain elements as they radioactively decay. These photons can be used to eradicate H. pylori as they are absorbed by the endogenous porphyrins.

The H pylori is killed by the blue/violet light when oxygen radicals are created damaging the bacteria's cell membrane. There are many light sources available to deliver high intensity light in many wavelengths. However, delivering multiple watts of power in a narrow wavelength band around 405 nm is not available readily from commercial light sources. External light sources emitting high power white light exist, but when all but the narrow 405+/−5 or 10 nm band is filtered out, the power is quite low. Blue lasers in this wavelength range exist, but with the exception of large experimental devices, their power is also low. Thus, although it may be possible to obtain a light source to deliver adequate power in the light band of interest, it is also of value to enhance the effectiveness of the light delivered. By the use of adjunct materials and other sensitizing means one can increase the effectiveness of any available light source. Examples of sensitizing materials include riboflavin, 5-amino levulinic acid (ALA), porfimer sodium, and motexafin lutetium.

It is possible to subject the bacteria to certain environmental stresses to make them more susceptible to the light delivered. H. pylori can be subjected to increased levels of oxygen so that the creation of oxygen radicals is more frequent, thereby creating more oxygen radicals for bacterial destruction. Bacteria are sensitive to their environment, and H. pylori is a sensitive bacterium. In vitro tests have revealed that the bacteria are sensitive to the level of iron available in the growth medium, the gas composition provided during growth, and even the length of time that the culture has been grown. Thus, modifying the local environment in the stomach can be used to facilitate the eradication by light by making the bacteria more fragile or susceptible. For example, techniques including ingestion or spraying of iodine or an iodine containing liquid like Lugol's solution, altering the pH levels, or increasing the temperature of the stomach, for example using hot water or some other means, can be used to compromise the bacteria's resistance to illumination.

It is well know that bacteria need iron for robust replication. Giving the patient an iron chelating agent decreases the free iron available thereby making the bacteria more susceptible to the light treatment. Alternatively, the bacteria may be more susceptible just after replication. Thus, providing a source of free iron may make it more susceptible to eradication through light treatment. These and other means for making the bacteria more susceptible to light treatment can be used.

The patient's gastric mucosa or resident H. pylori are stained directly with a fluorescent dye(s), which are then illuminated in vivo at the appropriate excitation wavelength for the dye. As the dye is chemically attached or bound to the object of interest, the effectiveness of the light for eradication of the H. pylori is enhanced. The dye can be swallowed, sprayed, painted on the surface, for example, using an endoscope, or delivered through an intravenous injection or ingested by the patient.

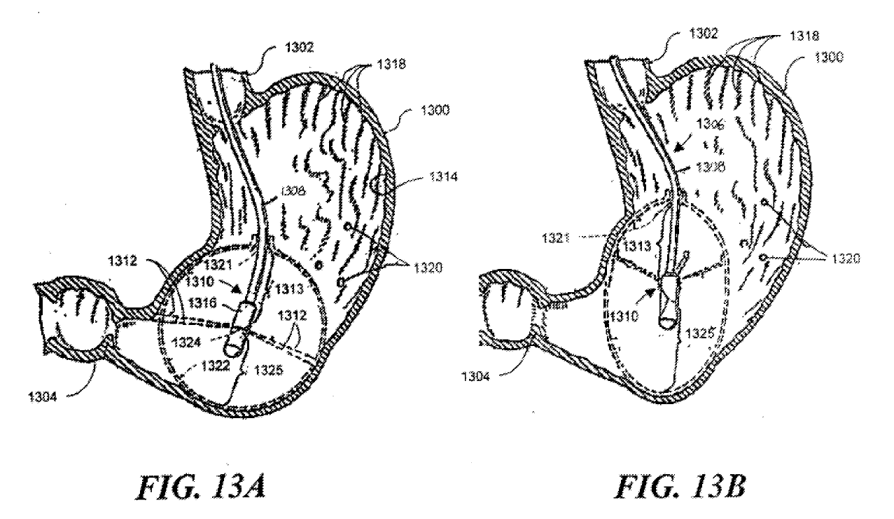

By way of illustrative example, referring to FIGS. 13A and 13B, one method of use in accordance with the present invention is shown for the treatment of H. pylori infections of the stomach 1300. The stomach 1300 is illustrated together with the esophagus 450a and the pyloric sphincter 1304. An instrument 1306 is provided including a flexible supporting cable or shaft 1308 with a delivery element, or distal light diffusing distribution head 1310. Visible light emanates from the distribution head 1310 as shown by rays 1312 that strike the adjacent lining of the stomach 1300 where the H. pylori infection thrives in the epithelium and mucous lining 1314. The head 1310 includes a diffuser of visible light 1316. It is contemplated that different types of maneuvering devices could be employed to position the head 1310 depending upon the particular site to be treated. In the embodiments showing the use of the instrument 1306 in the stomach 1300 and gastrointestinal system, it is beneficial for the shaft 1308 to be flexible, having a reduced diameter and a smoothed, or rounded forward end so that it can be easily introduced into the esophagus and stomach, either by itself or, if desired, through an appropriate flexible endoscope (not shown). In one particular embodiment, the shaft 1308 has an outer diameter of less than or equal to approximately 3 mm, allowing it to fit easily within a standard endoscope that typically has a working lumen diameter of about 3 mm. In other applications, the properties and dimensions of the shaft 1308 can vary to meet the requirements of the task.

For many disorders, rays 1312 forming an annular, or donut-shaped, visible light pattern is ideally suited for treatment. In order to achieve this pattern, passages and other exterior portions of the body should be dilated before and during treatment using light from the diffuser 1316. The stomach 1300 is very soft and, except after a meal, is in a collapsed state. Rugae or folds 1318 are generally present on its inner walls. In some instances, the stomach includes ulcers resulting from an H. pylori infection 1320.

In one preferred embodiment of the present invention an optional dilating balloon 1322 is optionally provided to dilate the interior region of the body, such as the stomach, thereby distending the stomach wall and hence spread the rugae 1318 apart, thus flattening the stomach wall. Having a flattened stomach wall facilitates generation of a uniform annular light pattern thereon by the head 1310. The balloon 1322 can also assist in positioning and holding the diffusing head 1310 in a desired location. One advantageous location is within a central position, substantially equidistant from all parts of the surrounding stomach wall. Such a positioning of the head 1310 leads to substantially the same dose of light reaching substantially all portions of the stomach 1300.

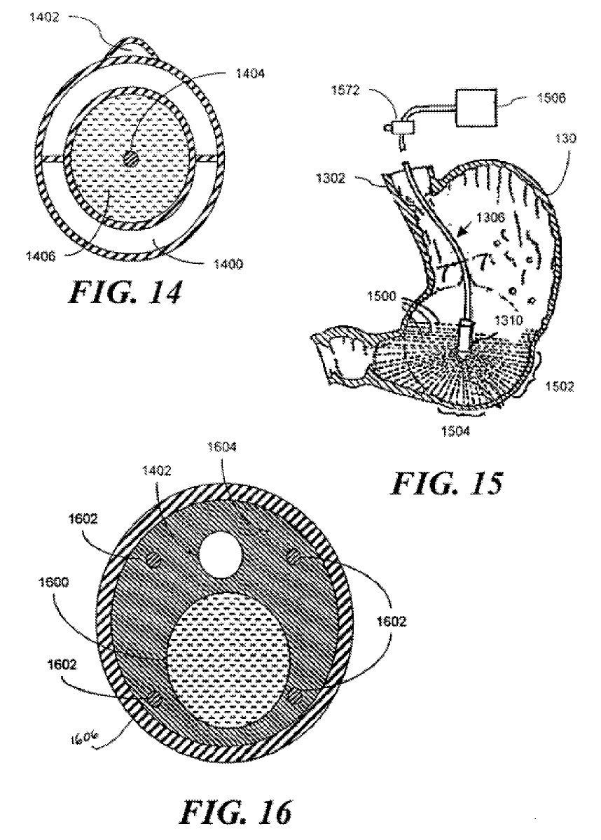

Using a light source placed within a patient's body (an internal light source), such as an incandescent bulb, without precautions, can lead to complications. For example, tissue damaging heat is generally produced at the filament of the bulb during a treatment procedure. Circulating a cooling substance, such as water, through the balloon's interior, serves to cool the light source and dissipate any potentially damaging heat. If desired, the balloon 1322 can be in fluid communication with a fluid loop 1400 (FIG. 14) disposed within the shaft 1308 to carry fluid from outside the body to the interior of the balloon 1322, and also providing a return path for the fluid. The fluid in the loop 1400 can circulate within the interior of the balloon 1322, thereby inflating the balloon 1322, and can be returned to the proximal portion of the shaft 1308 through the fluid loop 1400. A circulating pump can also be provided to circulate the fluid and maintain the pressure required to achieve a desired balloon size. Other methods and devices known in the field can also be used to circulate the fluid and inflate the balloon 1322.

Since it is generally desirable to provide independent control of the balloon size and cooling rate, a separate inflation lumen 1402 and port 42 are shown in FIGS. 14-16 in fluid communication with the balloon 1322. The fluid loop 1400 is positioned to circulate cooling fluid in heat conducting relationship with the diffusing head. The circulating action of the fluid loop 1400 can thus provide a constant cooling rate, regardless of the extent of balloon dilation. The separate inflation lumen 1402 can be coupled to a fluid source (not shown) of adjustable pressure for the balloon 1322 via the inflation lumen 1402. In one embodiment, the fluid loop 1400 and the inflation lumen 1402 are created using plastic extrusion techniques. This arrangement has the advantage of allowing a liquid, e.g., water, to be used in fluid loop 1400 for cooling and a gas, e.g., air, to be used for balloon inflation via lumen 1402 so that the light from the head 1508 is not substantially absorbed prior to reaching the stomach wall.

Different cooling mechanisms can also be used, such as expanding the balloon with an inflating fluid provided via lumen 1402. If a liquid is used to inflate the balloon instead of a gas such as air, the liquid, e.g., water or saline, can be supplied from a tank. A gas, however, is preferred for filling the balloon 1322, since it will have a negligible tendency to attenuate the light 1312 emitted from the energy supply head 1310 and will allow the balloon 1322 to inflate and deflate quicker and easier. The coolant is circulated separately through the fluid loop 1400.

The stomach in its relaxed state has a diameter of about 5-6 cm and is generally unable to accommodate a rigid structure. In one embodiment, the device of the present invention can be inserted by being passed through a standard flexible endoscope (not shown) that has a working lumen about 3 millimeters in diameter.

In some applications, such as use in the stomach, the diameter of the dilated balloon 1322 can vary with the pressure applied, so that the diameter of the balloon can be adjusted to fit the size of the patient's stomach or other passage. Therefore, an elastic balloon is particularly suited to gastric applications, where the elastic material will conform to the many surface features of the stomach and dilate the stomach more completely. However, in other applications, it can be desirable to employ an inelastic balloon with a fixed dilated diameter. It should be noted in FIG. 13A that the balloon 1322, when present, is secured to the flexible shaft 1308, e.g., by means of a suitable adhesive 1321 at a distance 1313 from source 1322 and also spaced from the radiation head 1310. The distal end of the balloon 1322 remains free and is spaced from the light diffuser by a distance 1325 that is equal to 1313. The distances 1313 and 1325 each equal the approximate radius of the balloon 1322 so as to locate the source 1324 of the light 1312 substantially at the center of balloon 1322, thus equalizing illumination in all directions. A round balloon is shown in FIG. 13A.

When used to radiate the walls of an interior passage of the body, according to one embodiment of the invention, the light transmission device can be placed within a standard endoscope, such as a laryngoscope or gastroscope. The light transmission device described herein is introduced into the passage to be treated. The light transmission device, etc., is then guided through the passage, using techniques known in the art, until it is positioned near the area to be illuminated. The site to be illuminated can be viewed through the endoscope, and the area around the device can be flushed using the endoscope, if necessary. The dilating balloon 1322 is then inflated by fluid, either liquid or gas, from the fluid pump to the desired diameter to expand the body cavity, in this case the stomach so as to hold the light transmission head 1310 in the desired location and spread the rugae 1318 apart thereby flattening the stomach wall and insuring a substantially uniform light illumination.

During a treatment operation, the external light source is energized and light is coupled to the flexible light guide. As the light impinges upon the wall of the body cavity, e.g., the stomach, the H. pylori living on the surface of the passage are killed and or debilitated as discussed above. In H. pylori infections, for example, the necrosis eliminates the bacterial cells and reduces inflammation as well as the biochemical results of inflammation, thereby preventing ulcers, gastritis and cancer. When the desired dosage has been delivered, the light source is turned off and the balloon 1322, when present, is deflated. The device is then withdrawn from the body. In order to treat H. pylori only the surface region of the epithelium needs to be irradiated.

According to the present invention, light radiation typically in the range of 5-200 Joules/cm², and most preferably 30-50 Joules/cm², can be applied. The treatment is typically structured to last about 3 to 15 minutes, and preferably lasting 4 to 8 minutes. The light transmission device can be repositioned by moving it from one part of the stomach to another, by translation and/or rotation, either continuously or intermittently during the course of light treatment, depending on the area requiring treatment.

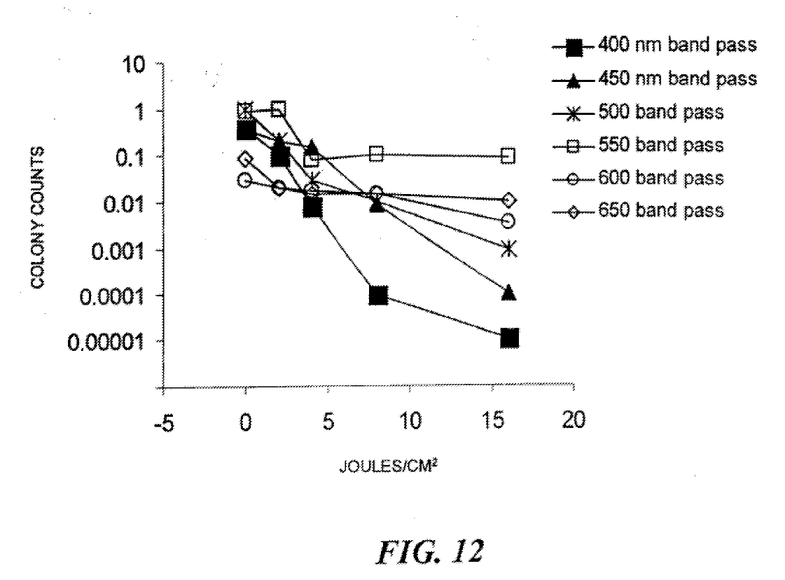

Test results have been plotted to illustrate the effectiveness of light at different wavelengths and intensities. FIG. 12 shows the H. pylori colony forming units along the vertical axis versus the light intensity along the horizontal axis. The lower colony counts reflect a more effective treatment. Additionally, multiple curves are plotted together with each curve representing test results for a illumination by light of a different wavelength. In general, all curves show increasing effectiveness with increasing intensity. Further, light in the blue/violet spectrum (400 nm to 450 nm), generally are more effective than the other wavelengths tested.

It will be noted that because the source of light transmission in the light diffusing head 1310 is at the center of the balloon 1322, all of the light rays 1312 traced from the head 1310 will be of substantially the same length when they strike the microorganisms. Such uniform illumination tends to assure uniform exposure to light wherever the light strikes the wall of the cavity that is being treated. Uniform light exposure is also aided through the flattening of the stomach wall that is accomplished by the expansion of the balloon 1322. Additionally, the expanded balloon 1322 locks or wedges the light transmission head 1310 in place within the stomach 1300 so that stomach contractions, which take place normally will not displace the instrument 1306. During use, the balloon 1322 is not expanded to the point where the blood supply to the epithelium lining the stomach is cut off, since oxygen is necessary in forming free radicals, which are important in the destruction of the microorganisms.

Refer now to FIGS. 14 and 15 illustrating a modified form of the invention in which the same numerals refer to corresponding parts already described. In this case, light rays 1500 are provided by the energy distribution head 1310, which is formed from a transparent material, e.g., glass or fused quartz. The light 1500 can be projected laterally 1502 and/or forwardly 1504 through the balloon 1322 striking the wall of the stomach 1300. The balloon 1322 holds the light energy distribution head 1310 in the desired position and also distends the wall of the stomach 1300 so as to spread out the rugae 1318 and thereby allow uniform exposure of the portion of the wall of the stomach that is being treated. As the light rays 1500 strike the columnar epithelium lining the stomach, the H. pylori infecting the cells is killed and/or debilitated.

The part of the stomach exposed to the light rays 1500 can be changed by the physician, either by moving the balloon 1322 and head 1310 along the length of the stomach 1300 toward the esophagus 1302, by changing the angle of the head 1310 with respect to the longitudinal axis of the stomach 1300 or by rotating the head 1310 about its longitudinal axis. The position of the instrument can also be confirmed using fluoroscopy or a CAT scan, if desired. In one embodiment, the delivery element 106, 206, 610 includes a radiopaque marking to facilitating tracking of its position using fluoroscopy during the procedure. A fiber optic bundle is deployed 1600 (FIG. 16), which extends from a light source 1506 (FIG. 15) through the entire length of the flexible shaft 1308 via the esophagus 1302 into the stomach 1300, so as to carry light from the source 1506 through the distribution head 1310 to a light reflector or diffuser, e.g., of conical shape, inside the distribution head 1310, which spreads the light rays 1500 so that they pass through the balloon 1322, striking the wall of the stomach 1300 to the side and in front of the distribution head 1310. As shown in FIG. 16, the inflation fluid for the balloon is supplied through a lumen 1402 as already described. The flexible shaft 1308 can be provided with a plurality of longitudinally extending, radially spaced-apart cables 1602 that are slidably mounted in the flexible body portion 1604 of the shaft 1308. Using a suitable commercially available steering mechanism for shortening or lengthening the cables 468, the distribution head 1310 can be made to point toward the right, left or up and down as directed by the physician to distribute the beam of visible: light to various parts of the stomach as desired. The shaft 1308 can be enclosed in a protective cover or sheath 1606, e.g., polypropylene plastic that will slide easily through the esophagus 1302.

The light source 1506 can comprise any suitable commercially available lighting source, e.g., a mercury vapor lamp, a blue/violet laser, etc.

To use the apparatus of FIGS. 15 and 16, the shaft 1308 and head 1310 are passed through the esophagus 1302 conventionally with the balloon 1322 in a collapsed position surrounding the head 1310. After the head 1310 is properly positioned in the stomach 1300 under the control of the physician, the balloon 1322 is inflated by passing a suitable fluid, e.g., air, through the inflation lumen 1402 until the balloon 1322 has expanded the stomach 1300 at the desired location, thereby distending the rugae 1318 so that the pockets otherwise present are spread out evenly over the surface of the balloon 1322. The light source 1506 is then turned on, causing the light to pass through the fiber optic bundle 1600 and out through the distribution head 1310. The distribution head 1310 and the balloon 1322 can then be repositioned in the stomach as desired to expose all of the infected areas or, alternatively, the control cables 1602 can be manipulated so as to point the head 1310 toward the areas of the stomach that require treatment. Observations can be carried out by means of a viewing port and eyepiece 1572 of known construction or through a separate endoscope (not shown) that is passed through the esophagus 1302 into the stomach 1300 alongside the flexible shaft.

By way of illustrative example, referring to FIGS. 19A-19C, a lamp 2000 can include any suitable lamp for producing visible light to kill and/or debilitate pathogenic bacteria. For example, the lamp 2000 can be an incandescent lamp, such as a mercury vapor lamp, or a flash lamp formed from fused quartz lamp, such as a xenon arc flash lamp. Further, the lamp 2000 can be made to operate in a pulsed mode flashing periodically at selected timed intervals. Additionally, the pulsed source can be a laser emitting at a wavelength of light effective in the treatment of the bacteria. One preferred lamp comprises a filtered short-arc xenon lamp as a light source for producing blue/violet light. While light at various wavelengths can be used, one particularly effective range is blue-violet light having wavelengths in and about 400-450 nm. Good results have been obtained in debilitating select porphyrin producing bacteria with a mercury vapor lamp producing filtered light between about 400-450 nm, with 405 nm being optimal for H pylori bacteria.

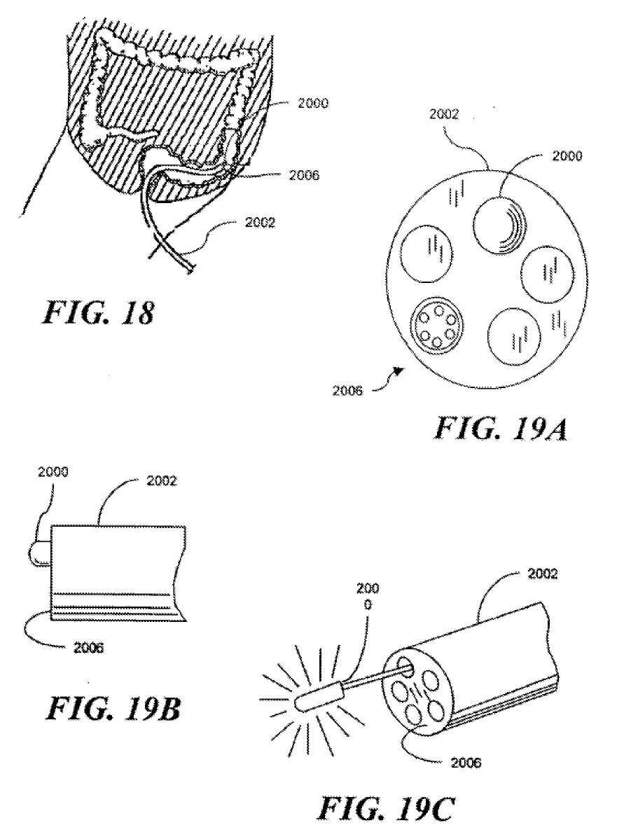

In addition to ailments of the stomach, bacteria have been implicated in causing certain intestinal disorders, such as Crohn's disease and inflammatory diseases of the bowel. Billions of many different types of bacteria proliferate normally in the bowel. The body, however, sometimes cross-reacts to either pathogenic or normal bacteria. Occasionally, after sensing the presence of normal bowel flora, the body attacks one or more of the bowel flora species as a pathogen, setting up a chronic inflammatory state, which makes the patient feel sick. Other gastrointestinal infections are caused by H. pylori as described above. To cure these conditions, in accordance with the present invention as shown in FIG. 18, microorganisms in the colon or other parts of the digestive tract are also killed and/or debilitated by visible light. Generally, only those bacteria producing endogenous porphyrins will be effected by the visible light treatment. Thus, this treatment is a selective approach for treating this group of bacteria.

Refer now to FIGS. 19A-19C, which illustrate in more detail the construction of the lower end of the shaft 2002 of the endoscope 2004. To protect the lamp 2000 while the shaft 2002 of instrument 2004 is being inserted into a body cavity, the lamp 2000 is withdrawn into the shaft 2002 as shown in FIG. 19B by means of a handle so that the lamp 2000 is either completely or at least partially recessed inside the lower shaft's end 2006 However, when the lamp 2000 is to be used, it is extended by the surgeon to a deployed position as shown in FIG. 19C. In the extended position, the lamp 2000 emits blue-violet light in all directions.

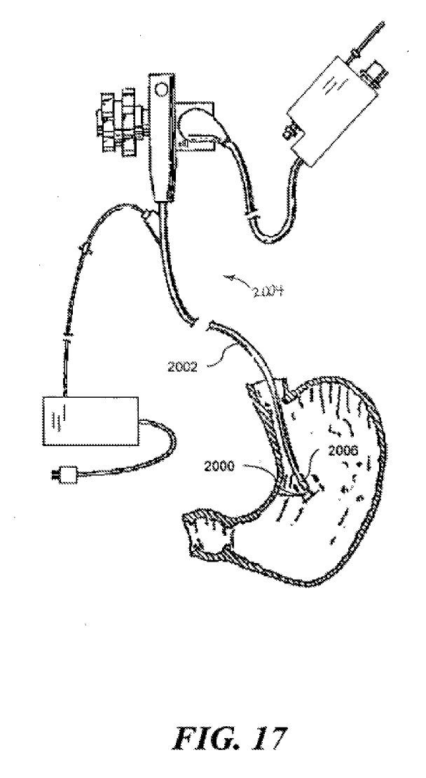

The invention will be better understood by reference to the following examples. Following symptoms, including stomach discomfort, “heart burn,” and/or pain, a tentative diagnosis by the physician of stomach ulcers is made, which is later confirmed by an endoscopic examination. The diagnosis can then be further confirmed with standard enzymatic tests to detect the presence of H. pylori. Upon detection of H. pylori, treatment using the present invention can commence. Following standard sedation, the shaft 2002 of the endoscope 2004 is inserted through the esophagus (FIG. 17). The head or tip end 2006 of the shaft 2002 is then positioned as required under the supervision of the physician and the power supply 1009 is turned on, thereby activating the computer contained in the power supply 1009 and causing a capacitor to discharge periodically through the mercury vapor or xenon arc lamp 2000, e.g., once every five seconds until treatment is concluded. The lamp 2000 is repositioned as necessary to provide adequate treatment to all of the affected areas, until the bacteria are either killed or incapacitated. The instrument 2004 is then withdrawn. A light-sensitizing medication can optionally be administered to the patient to enhance the desired effect. For example, the light sensitizing medication can cause the light to be preferentially absorbed by the bacteria, rather than by human cells. Any suitable light-sensitizing medicine can be used, such as any of the suitable protoporphyrin compounds known to those skilled in the art for preferentially absorbing the light so as to provide a more effective bacteriocidal action.

In some embodiments, the light source, in addition to emitting visible light, can emit electromagnetic radiation having wavelengths outside of the visible spectrum. In one embodiment, the light source includes electromagnetic radiation having wavelengths in the ultraviolet spectrum. In another embodiment, the light source includes electromagnetic radiation having wavelengths in the infrared spectrum. In providing a light source having the desired emission spectrum, it is possible to combine multiple light sources, as described in relation to FIG. 2, whereby each light source emits light at a respective range of wavelengths. For example, a visible light source can be coupled together with an ultraviolet light source. Additionally, for embodiments using light emitting elements, combinations of elements can be provided, whereby each element of the combination emits light at a respective range of wavelengths.

Having described certain embodiments of the invention, it will be apparent to those of ordinary skill in the art that other embodiments incorporating the concepts disclosed herein may be used without departing from the spirit and scope of the invention. The described embodiments are to be considered in all respects as only illustrative and not restrictive.

What is claimed is:

1. An apparatus for killing or debilitating pathogenic microorganisms within a patient's body, the apparatus comprising:
a light source external to the body emitting electromagnetic radiation having wavelengths within the visible spectrum;
a light guide having a proximal end optically coupled to the light source and a distal end dimensioned for insertion into a patient's body, the light guide transferring electromagnetic radiation having wavelengths within the visible spectrum therethrough; and
a delivery element optically coupled to the distal end of the light guide for directing to a location with a patient's body electromagnetic radiation having wavelengths within the visible spectrum.

2. The apparatus of claim 1, wherein the microorganisms are bacteria.

3. The apparatus of claim 2, wherein the bacteria are H. pylori bacteria.

4. (canceled)

5. The apparatus of claim 1, wherein the light source is selected from the group consisting of a laser, a laser diode, a light emitting diode, a lamp, and combinations thereof.

6. (canceled)

7. The apparatus of claim 1, further comprising an adapter optically coupled between the light source and the proximal end of the light guide.

8. The apparatus of claim 7, wherein the adapter is selected from the group consisting of a lens, a prism, a mirror, a fiber optic splice, a 1:N optical coupler, a connector, and combinations thereof.

9. The apparatus of claim 1, wherein the light guide is selected from the group consisting of single strand fiber optic cable, multi strand fiber optic bundle, a gas-filled channel, a fluid-filled channel, a sequence of reflectors, and combinations thereof.

10. The apparatus of claim 1, wherein the delivery element is selected from the group consisting of a lens, a prism, a mirror, a balloon, gas, liquid, fluid sprays, fiber fountains, frustrated total internal reflection pads, optically transmissive coatings, optically active materials, and combinations thereof.

11. A method for killing or debilitating pathogenic microorganisms within a patient's body, the method comprising the steps of:
providing a light source external to the body, the light source emitting electromagnetic radiation having wavelengths within the visible spectrum;
optically coupling the electromagnetic radiation into a light guide;
directionally coupling the electromagnetic radiation from the light guide to a location with a patient's body.

12. The method of claim 11, wherein the microorganisms are H. pylori bacteria.

13. (canceled)

14. The method of claim 11, further comprising the step of enlarging the location within the patient's body.

15. (canceled)

16. The method of claim 11, further comprising the step of sensitizing the pathogenic microorganisms to the directed electromagnetic radiation.

17. An apparatus for killing or debilitating pathogenic microorganisms within a patient's body, the apparatus comprising:
a light source dimensioned for insertion into a patient's body, the light source emitting electromagnetic radiation having wavelengths within the visible spectrum; and a delivery element optically coupled to the light source, for delivering a portion of the coupled visible light to a location within a patient's body.

18. (canceled)

19. The apparatus of claim 17, further comprising an energy source external to the body, wherein the energy source energizes the light source.

20. The apparatus of claim 19 further comprising a tether coupled between the light source and the energy source for coupling energy therebetween.

21. (canceled)

22. The apparatus of claim 17, wherein the delivery element comprises the light source.

23. A method for killing or debilitating pathogenic microorganisms within a patient's body, the method comprising the steps of:
providing a light source dimensioned for insertion into a patient's body, the light source emitting electromagnetic radiation having wavelengths within the visible spectrum;
energizing the light source; and
directionally coupling at least a portion of the emitted electromagnetic radiation to a location containing pathogenic microorganisms within a patient's body.

24. The method of claim 23, wherein the microorganisms are H. pylori bacteria.

25. (canceled)

26. (canceled)

27. The method of claim 23, further comprising the step of sensitizing the pathogenic microorganisms to the directed electromagnetic radiation.

28. An apparatus for killing or debilitating pathogenic microorganisms within a patient's body, the apparatus comprising:
a light-emitting material emitting electromagnetic radiation having wavelengths within the visible spectrum; and
means for directing at least a portion of the light-emitting material to a location within a patient's body, the location containing pathogenic microorganisms.

29-33. (canceled)
